(19) United States
(12) Patent Application Publication
(10) Pub. No.: US 2023/0025155 A1
(43) Pub. Date: Jan. 26, 2023

(54) Fungus Strain Having Decreased Viscosity

(71) Applicant: IFP Energies Nouvelles, Rueil Malmaison (FR)

(72) Inventors: Frédérique Bidard-Michelot, Rueil Malmaison (FR); Etienne Jourdier, Rueil Malmaison (FR); Fadhel Ben Chaabane, Rueil Malmaison (FR); Thiziri Aouam, Rueil Malmaison (FR); Sabine Prigent, Rueil Malmaison (FR)

(73) Assignee: IFP Energies Nouvelles, Rueil Malmaison (FR)

(21) Appl. No.: 17/780,088

(22) PCT Filed: Dec. 4, 2020
(86) PCT No.: PCT/FR2020/052282

§ 371 Date: May 26, 2022

(30) Foreign Application Priority Data
Dec. 4, 2019 (FR) FR1913766

(51) Int. Cl. C12P 7/08; C12N 1/14; C12N 15/90; C12N 9/42

(52) U.S. Cl. CPC C12P 7/08; C12N 1/145; C12N 15/90; C12N 9/2437; C12Y 302/01004; C12R 2001/885

(57) ABSTRACT

The invention relates to a strain of fungus having a reduced viscosity, wherein the ID 78713 (GEL3) gene has been invalidated. The invention also relates to the different uses of this strain, as well as to the method of genetic modification.

The present invention relates to a strain of fungus, in particular a filamentous fungus, having a reduced viscosity, in which the ID78713 (GEL3) gene has been invalidated. The invention also relates to the different uses of this strain, as well as to the genetic modification method enabling a strain according to the invention to be obtained.

### CONTEXT OF THE INVENTION

Strains of fungi, particularly filamentous fungi, such as the Trichoderma reesei fungus, are mainly used today for the production of enzymes. These enzymes, for example cellulases, are in fact used to hydrolyze cellulosic or lignocellulosic biomass into simple sugars. Therefore, the enzymes produced by filamentous fungi are useful in the production chains of second-generation biofuels or of bio-sourced products coming from (ligno)cellulosic biomass sugars.

In order to improve the production of second-generation biofuels or bio-sourced products, improving the production of cellulases has been conceived.

For example, patent EP 448 430 B1 describes an optimized industrial production of cellulases by Trichoderma reesei. This production is carried out in the fed-batch protocol (supply without withdrawal) by using a feed solution containing lactose as the sugar inducing the production of cellulases. This fermentation method comprises two steps: a first step of fungal growth in the presence of an excess of a carbon source and a second step of enzyme production by means of the addition of an inducer into the medium with an optimized flow rate. These steps are carried out in a liquid medium in stirred bioreactors and in the presence of oxygen since the fungus is an obligate aerobe. Another example of an optimized cellulase production method is described in patent EP 2 744 899 B1.

In addition to optimizing cellulase production methods, genetically modifying fungal strains in order to modify their production capacity has also been conceived. Therefore, patent application EP 18 197 010.4 describes a strain of Trichoderma reesei that hyper-produces cellulolytic enzymes.

Fermentation methods and strains useful for improving the production of cellulolytic enzymes by filamentous fungi and, as a result, bio-sourced products, and also second-generation biofuels have already been described in the prior art.

Nevertheless, the morphology of industrial filamentous fungi has significant consequences on the design and implementation of fermentation methods because it increases the viscosity of the culture medium. Thus, viscosity has a negative effect on the transfer of oxygen and limits the maximum concentration of fungus that can be targeted at the end of the growth step (and this, consequently, limits the quantity of cellulolytic enzymes that can be produced).

In order to mitigate this problem, agitating the fermenters at higher speeds has first been conceived. However, increasing the agitation is not an ideal solution since this leads to increased energy expenditure and to increased shear stress on the mycelium, which also has a negative impact on the production of cellulolytic enzymes.

Altering the viscosity of filamentous fungi by modifying their genome has also been conceived. These modifications mainly consist of invalidating genes, the absence of proteins or enzymes produced by these genes leading to a change in cell function (Bodie and Pratt 2012; Dodge, Virag and Ward 2012).

Thus, in application PCT/US2012/034379, Bodie and Pratt conceived of invalidating the mpgl gene in order to alter the viscosity of a fungus strain. Additional invalidations have also been conceived (among the sfb3, sebl, gas1, crzl or tps2 genes).

For their part, in application PCT/US2011/049164, Dodge, Virag and Ward conceived of invalidating the sfb3 gene.

Fungal strains that have been genetically modified in order to alter their viscosity compared to non-mutant strains (i.e. parent strains) have already been described in the prior art. However, it should be noted that the prior art is silent regarding the specific improvement in viscosity values compared to the parent strains.

In addition, there is always a need for novel, less-viscous strains. Moreover, there is always a need for novel, better-performing strains enabling better growth of the fungus compared to parent strains.

### BRIEF DISCLOSURE OF THE INVENTION

Thus, the present invention is based on the unexpected results of the inventors who demonstrated that invalidating the ID78713 (GEL3) gene in a strain of fungus (belonging, in particular, to the class of sordariomycetes), enables a strain to be obtained having a drastically reduced viscosity compared to a parent strain in which said ID78713 (GEL3) gene was not invalidated. The inventors have in fact demonstrated that invalidating the ID78713 (GEL3) gene altered the viscosity of said fungi, particularly filamentous fungi. Thus, the use of such a strain enables the viscosity of a fermentation must at iso-concentration of fungi (i.e. for a same concentration of fungi in the must) to be reduced, which thereby enables the costs of the methods for fermenting filamentous fungi to be lowered. This also enables the productivity of the method to be increased by obtaining higher fungal concentrations. In fact, enzyme volume productivity is proportional to fungal concentration and to the specific rate of enzyme production. Thus, having a less-viscous strain makes it possible to reach higher fungal concentrations and to increase the enzyme productivity of the method.

The inventors of the present invention are thus the first to have demonstrated that the (GEL3) gene could influence the phenotype viscosity of fungal strains. In fact, the ID78713 (GEL3) gene encodes a protein from the glycoside hydrolase family 72, particularly 1,3-β-glucanosyltransferase. The document Mouyna et al., Microbiology (1998), 144, 3171-3180, describes obtaining a mutant of Aspergillus fumigatus (a fungus belonging to the class of Eurotiomycetes), in which the gene encoding 1,3-β-glucanosyltransferase BGT1 is invalidated; nevertheless, the authors conclude that the variant strain does not present a different phenotype compared to the parent strain.

The present invention thus relates to a strain of fungus in which the ID78713 (GEL3) gene has been invalidated.

The present invention also relates to a method of genetically modifying a fungal strain according to the invention, comprising a step of invalidating the ID78713 (GEL3) gene.

The present invention also relates to a method of producing a fungal biomass, comprising a step of culturing a fungal strain according to the invention in a culture medium comprising an appropriate substrate.

The present invention also relates to a method of producing proteins of interest, particularly enzymes, comprising a step of culturing a fungal strain according to the invention in a culture medium comprising an appropriate substrate.

The present invention further relates to a method of producing bio-sourced products from cellulosic or lignocellulosic substrates, comprising a step of using the fungal strain according to the invention to produce cellulolytic enzymes.

The present invention also relates to a method of producing a biofuel from cellulosic or lignocellulosic substrates, comprising a step of using the fungal strain according to the invention to produce cellulolytic enzymes.

The present invention also relates to different uses of the strain according to the invention for the production of proteins of interest, for the hydrolysis of cellulose or lignocellulose into glucose, for the production of bio-sourced products from cellulosic or lignocellulosic substrates, or for the production of biofuel.

Lastly, the present invention relates to the use of a fungal strain according to the invention to improve the properties of a compatible strain, particularly an industrial strain.

### DISCLOSURE OF THE INVENTION

In a first aspect, the present invention thus relates to a strain of fungus in which the ID78713 (GEL3) gene has been invalidated. Thus, in the strain according to the invention, the ID78713 (GEL3) gene is no longer functional. This also means that in the strain according to the invention, the ID78713 (GEL3) gene is invalidated. The present invention thus relates to a variant strain of fungus in which the ID78713 (GEL3) gene has been invalidated. In other words, this means, for example, that in the strain according to the invention, the protein corresponding to the ID78713 (GEL3) gene is not produced. Alternatively, the protein corresponding to the ID78713 (GEL3) gene may be produced, but is not functional.

According to the invention, the term “variant strain” is understood to refer to a strain that has been genetically modified compared to a parent strain. According to the invention, the term “parent strain” is thus understood to refer to a strain from which the variant strain is descended or derived, and in which the ID78713 (GEL3) gene has not been invalidated. The strain according to the invention thus corresponds to a variant strain derived from a parent strain, said variant strain having a reduced viscosity compared to the parent strain and said variant strain comprising at least one genetic modification corresponding to the invalidation of the ID78713 (GEL3) gene.

According to the invention, the term “functional gene” is understood to refer to, in particular, a gene that enables a functional protein to be produced.

According to the invention, the term “functional protein” is understood to refer to, in particular, a protein that has an activity: for example, with the protein encoded by the ID78713 (GEL3) gene, a glucoside hydrolase activity.

According to the invention, said fungal strain has a reduced viscosity compared to a parent strain in which the ID78713 (GEL3) gene has not been invalidated. According to a preferred embodiment, the fungal strain has a viscosity at least 3 times lower compared to the parent strain, more specifically at least 8 or 10 times lower.

The expression “a reduced viscosity compared to the parent strain” means that the variant strain has a viscosity that is lower than that of the parent strain. The person skilled in the art knows that the viscosity of the variant strain according to the invention and that of the parent strain should be compared for a same fungal concentration in the fermentation must.

According to the invention, viscosity is preferentially measured by the method described in Example 2, also described in the publication Hardy et al., Rhéologie, Vol. 27, 43-48 (2015), or else by using the following parameters (called, for example, Test A):
the shaft (rotor) used is an impeller with a diameter of 38 mm, a height of 32 mm, a pitch of 29 mm and with a ribbon 8 mm in width;
a cup (stator) of 45 mm in inner diameter;
a vertical space between the rotor and stator of 500 μm; and the following protocol:
filling the cup with 70 mL of a medium for which the viscosity is to be measured;
measuring the viscosity by logarithmic shear rate sweeps of between 4 s⁻¹ and 100 s⁻¹, and then 100 s⁻¹ to 4 s⁻1, at a temperature of 27° C.;
optionally, carrying out measurements in duplicate.

Preferentially, the viscosity according to the invention is measured by using a TA Instruments AR 2000 rheometer, particularly by logarithmic shear rate sweeps of between 4 and 100 s⁻¹ at a temperature of 27° C., even more preferentially by using two-way sweeping (from 4 s⁻¹ to 100 s⁻¹ and then from 100 s⁻¹ to 4 s⁻¹). According to one embodiment, the strain according to the invention has a viscosity of approximately 1.5 Pa·s, as measured at 27° C., using a TA Instruments AR 2000 rheometer with logarithmic shear rate sweeps of 5 s⁻¹, when the fungal concentration is approximately 35 g/L. “Fungal concentration” is understood to refer to the concentration in the medium in which the viscosity is measured. Typically, the medium corresponds to a fermentation must. According to the invention, the term “approximately” means that the values should not be regarded as strict values. Thus, “approximately 1.5 Pa·s” is understood to refer to values between 1.45 Pa·s and 1.55 Pa·s, and “approximately 35 g/L” is understood to refer to values between 34.5 g/L and 35.5 g/L.

According to the invention, and in another particular embodiment, the strain according to the invention has a viscosity that is reduced by at least 50% compared to a parent strain. According to the invention, the term “at least 50%” means all values between 50% and 100%, particularly the values of 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%. Preferentially, the strain according to the invention has a viscosity that is reduced by at least 65% compared to a parent strain. The person skilled in the art knows how to calculate a percentage decrease. Such a rate of decrease may, for example, be calculated according to the following formula: ((final value−initial value)/initial value)*100.

The GEL3 gene (also called ID 78713 in the Trichoderma reesei reference genome, see https://www.uniprot.org/uniprot/G0RL27 encodes a protein that belongs to the glycoside hydrolase family 72. These enzymes are, for example, β-1,3-glucanosyltransglycosylases that participate in cell wall biogenesis in fungi. According to the invention, the term ID 78713 is preferred over the term GEL3.

According to the invention, the ID78713 (GEL3) gene is represented by SEQ ID NO: 2, but may also correspond to a variant of this gene or to an orthologous gene. Preferentially, the ID78713 (GEL3) gene is only represented by SEQ ID NO: 2.

According to the invention, “a gene variant or an orthologous gene” is understood to refer to a gene that also encodes a protein that belongs to the glycoside hydrolase family 72. The variant of the ID78713 (GEL3) gene or an orthologous gene of the ID78713 (GEL3) gene is typically represented by a sequence having at least 80% identity with the gene of SEQ ID NO: 2. The variant of the ID78713 (GEL3) gene or an orthologous gene of the ID78713 (GEL3) gene thus corresponds to a gene derived from the sequence represented by SEQ ID NO: 2. More specifically, the variant of the ID78713 (GEL3) gene or an orthologous gene of the ID78713 (GEL3) gene is represented by a sequence having at least 90% identity with the SEQ ID NO: 2 gene, particularly at least 95%, preferably at least 98% or 99%.

The ID78713 (GEL3) gene is represented by SEQ ID NO: 2, and the protein encoded by the ID78713 (GEL3) gene is represented by SEQ ID NO: 3. Thus, the variant of the ID78713 (GEL3) gene or an orthologous gene of the ID78713 (GEL3) gene encodes either the protein of SEQ ID NO: 3, or encodes a sequence having at least 80% identity with said SEQ ID NO: 3, particularly at least 90%, more specifically at least 95%, 98% or 99%.

According to the invention, the term “at least 80%” means all values between 80% and 100% inclusive, particularly the values of 80%, 81%, 82%, 83%, 84%, 85%, 86%, 87%, 88%, 89%, 90%, 91%, 92%, 93%, 94%, 95%, 96%, 97%, 98%, 99% and 100%. The person skilled in the art knows how to calculate an identity percentage between two sequences. For example, according to the invention, the identity percentage of a given sequence compared to SEQ ID NO: 2 or SEQ ID NO: 3 is understood to refer to the identity percentage over the total length of the sequences. The percentage thus corresponds to the number of identical nucleotides/residues between this given sequence and SEQ ID NO: 2 or 3 divided by the number of nucleotides or residues in the longer of the two sequences.

Thus, in one embodiment, in said strain according to the invention, the invalidated ID78713 (GEL3) gene corresponds to a gene represented by SEQ ID NO: 2 or to a gene having at least 80% identity with the gene of SEQ ID NO: 2, particularly at least 90% and more specifically at least 95%. In an even more preferred embodiment, in said strain according to the invention, the invalidated ID78713 (GEL3) gene corresponds to a gene represented by SEQ ID NO: 2. According to this last embodiment, the strain according to the invention thus comprises a deletion of the gene encoding the protein represented by SEQ ID NO: 3.

In a preferred aspect, in the strain according to the invention, the ID78713 (GEL3) gene has been invalidated by mutagenesis or by homologous recombination. According to the invention, the invalidation consists of a deletion of all or part of the ID78713 (GEL3) gene. Preferentially, in the strain according to the invention, the ID78713 (GEL3) gene has been invalidated by using an invalidation cassette. Still more preferentially, said invalidation cassette is represented by SEQ ID NO: 1, and is used in particular in a fungus belonging to the Trichoderma reesei species.

Mutagenesis is a commonly-used technique in genetic engineering. It aims to voluntarily introduce mutations into DNA in order to create genetically modified genes. According to the invention, mutagenesis more specifically is understood to refer to site-directed mutagenesis. In fact, site-directed mutagenesis enables identified mutations to be introduced into a specific gene. For this purpose, the DNA of interest (here the ID78713 (GEL3) gene) containing the mutations is synthesized and then introduced into the cell to be mutated, typically by using a vector, where the DNA repair mechanism integrates it into the genome.

Homologous recombination is a commonly-used technique in genetic engineering that consists of an exchange between DNA molecules, typically by using a vector.

The term “vector” is understood to refer to any DNA sequence in which it is possible to insert foreign nucleic acid fragments, the vectors enabling foreign DNA to be introduced into a host cell. Examples of vectors are plasmids, cosmids, yeast artificial chromosomes (YAC), bacterial artificial chromosomes (BAC), bacteriophage P1-derived artificial chromosomes (PAC), and virus-derived vectors. The vector according to the invention enables a mutation or a deletion to be introduced.

In a preferred embodiment, said invalidation cassette comprises three DNA fragments:
(1) a region upstream from the target gene,
(2) a selection marker, and
(3) a region downstream from the target gene,

In the case of the present invention, “target gene” is understood to refer to the ID78713 (GEL3) gene. The regions upstream and downstream from the target gene are two recombination elements, one at each end of the gene, and are necessary for precisely targeting the sequence to be invalidated.

According to the invention, the region upstream from the target gene (i.e. sequence 5′ upstream from the ID78713 (GEL3) gene) is represented in particular by the sequence SEQ ID NO: 10.

According to the invention, the region downstream from the target gene (i.e. sequence 3′ downstream from the ID78713 (GEL3) gene) is represented in particular by the sequence SEQ ID NO: 12.

The expression “selection marker” is understood to refer to a gene whose expression gives the cells containing it a characteristic allowing them to be selected. In fact, the use of a selection marker enables the identification of cells that have integrated a genetic modification compared to those that have not integrated the modification. This is, for example, an antibiotic resistance gene, particularly the hygromycin antibiotic resistance gene hph, as represented by SEQ ID NO: 11.

More specifically, according to the invention, the invalidation cassette is preferentially composed of a resistance gene placed under the control of a promoter and of a terminator, with flanking regions 5′ and 3′ upstream and downstream from the ID78713 (GEL3) gene. Even more preferentially, according to the invention, the invalidation cassette is composed of a hygromycin antibiotic resistance gene hph placed under the control of the GPDa promoter and the TRPc terminator (Punt and van den Hondel, 1992), with flanking regions 5′ and 3′ upstream and downstream from the ID78713 (GEL3) gene. According to the invention, said invalidation cassette may be operably linked to a promoter, a terminator or any other sequence necessary for its expression in a host cell.

The invalidation cassette may be amplified according to conventional techniques well-known to the person skilled in the art, typically by a method selected among standard cloning, fusion PCR, or else in vivo PCR cloning. Preferentially, this invalidation cassette is amplified by PCR, particularly by using the sequences represented by SEQ ID NO: 4 and SEQ ID NO: 5. The invalidation cassette is then introduced by recombination into a strain, particularly a Trichoderma reesei strain, that does not express a selection marker gene. The person skilled in the art may easily identify the selection marker genes that are relevant for the implementation of the invention. After culturing, the variant/mutant strains that had incorporated the invalidation cassette are selected based on the expression or non-expression of the selection marker; the clones that had been transformed are the ones expressing said selection marker. These are strains according to the invention. Preferentially, mutant strains are identified by using primers of SEQ ID NO: 6, SEQ ID NO: 7, SEQ ID NO: 8 and SEQ ID NO: 9. These genetic recombination techniques are well known to the person skilled in the art. In a preferred aspect, according to the invention, the fungus is a filamentous fungus. Even more preferentially, the filamentous fungus is selected among the classes of orbiliaceae, pezizomycetes, dothideomycetes, eurotiomycetes, lecanoromycetes, leotiomycetes, sordariomycetes, and saccharomycetes. Advantageously, the filamentous fungus according to the invention belongs to the class of sordariomycetes, particularly to the genus Trichoderma, and more specifically to the species Trichoderma reesei.

When the fungus belongs to the species Trichoderma reesei, according to the invention, the parent strain of Trichoderma reesei may be the QM6a strain (deposited under ATCC number 13631), or else a strain issued from natural isolate QM6a (particularly obtained by random or directed mutagenesis), such as the strain Rut-C30 (deposited under ATCC number 56765), the strain deposited under CNCM number I-5221 (deposited on 3 Aug. 2017 at the National Collection of Cultures and Microorganisms (CNCM, Collection Nationale de Cultures de Microorganismes), of the Pasteur Institute, 25 rue du Docteur Roux, F-75724 Paris cedex 15), the strain NG14 (deposited under ATCC number 56767) or else the strain QM9414 (deposited under ATCC number 26921). Typically, the strains according to the invention present a low viscosity phenotype while still maintaining their ability to produce proteins of interest.

In a second aspect, the invention also relates to a method of genetically modifying a fungal strain according to the invention, such as mentioned above, comprising a step of invalidating the ID78713 (GEL3) gene. The method of genetically modifying a strain according to the invention thus allows the obtaining of fungal strains that are less viscous compared to the parent fungal strain. This is why the strain according to the invention can be considered to be a variant of the parent fungal strain. Thus, during its growth, the fungal strain according to the invention generates a lower viscosity compared to the parent strain for a given shear rate, at a same biomass concentration, and at a same temperature.

Preferentially, in said genetic modification method according to the invention, the step of invalidating the ID78713 (GEL3) gene is carried out using a mutagenesis or homologous recombination step. Still more preferentially, in said genetic modification method according to the invention, the step of invalidating the ID78713 (GEL3) gene is carried out by using an invalidation cassette such as mentioned above, in particular as represented by SEQ ID NO: 1, in a fungus belonging to the species Trichoderma reesei.

In a third aspect, the present invention also relates to a method of producing a fungal biomass, comprising a step of culturing a fungal strain according to the invention in a culture medium comprising an appropriate substrate. This step thus enables the growth of the fungal strain according to the invention. The person skilled in the art would know the appropriate substrates for growing fungal strains.

In a fourth aspect, the present invention also relates to a method of producing proteins of interest, particularly enzymes, comprising a step of culturing a fungal strain according to the invention in a culture medium comprising an appropriate substrate. The invention thus relates to the use of a fungal strain according to the invention for the production of proteins of interest.

Advantageously, said method thus comprises a growth phase of growing a fungal strain according to the invention, and then a phase of growing and producing proteins of interest by said strain. Still more preferentially, said growth phase is carried out in the presence of a growth substrate and said phase of growing and producing proteins of interest is carried out in the presence of an inducing substrate. The growth substrate and the inducing substrate are preferably carbon substrates.

Thus, the carbon growth substrate is preferably selected among lactose, glucose, xylose, residues obtained after ethanol fermentation of monomeric sugars of enzymatic hydrolysates of cellulosic biomass, and/or crude extracts of water-soluble pentoses from the pretreatment of a cellulosic biomass.

The inducing carbon substrate is thus preferably selected among lactose, cellobiose, sophorose, residues obtained after ethanol fermentation of monomeric sugars of enzymatic hydrolysates of cellulosic biomass, and/or crude extracts of water-soluble pentoses from the pretreatment of a cellulosic biomass.

According to the invention, the proteins of interest are all proteins that can be produced by a fungus, naturally or by genetic modification (for example, after transformation by using an appropriate vector).

Advantageously, the proteins of interest according to the invention are enzymes, particularly cellulolytic enzymes such as cellulases or hemicellulases. Preferably, the enzymes are cellulases. According to the invention, the term “cellulases” is understood to more specifically refer to enzymes selected among the endoglucanases, the exoglucanases and the glucosidases, and more specifically, β-glucosidase. The term “cellulase” more specifically refers to an enzyme adapted to the hydrolysis of cellulose and enabling the microorganisms (such as Trichoderma reesei) that produce them to use cellulose as a source of carbon, by hydrolyzing this polymer into simple sugars (glucose). The production of cellulases by a strain according to the invention, particularly Trichoderma reesei, may be determined by any usual techniques available to those skilled in the art, or else by the techniques described in patents EP 448 430 B1 or EP 2 744 899 B1.

A correlation between total secreted proteins and cellulases may be made, because in T. reesei, the principal exoglucanases (CBHI, CBHII) and endoglucanases (EGI, EGII) can represent up to 90% of the total quantity of secreted proteins (see, for example, Markov, A. V., Gusakov, A. V., Kondratyeva, E. G., Okunev, O. N., Bekkarevich, A. O., and Sinitsyn, A. P. (2005). New Effective Method for Analysis of the Component Composition of Enzyme Complexes from Trichoderma reesei. Biochemistry (Moscow) 70, 657-663).

In a fifth aspect, the present invention also relates to a method of producing bio-sourced products from cellulosic or lignocellulosic substrates, comprising a step of using the fungal strain according to the invention to produce cellulolytic enzymes. The invention thus also relates to a method of using a fungal strain according to the invention for producing bio-sourced products from cellulosic or lignocellulosic substrates.

In a sixth aspect, the present invention relates to a method of producing a biofuel from cellulosic or lignocellulosic substrates, comprising a step of using the fungal strain according to the invention to produce cellulolytic enzymes. The invention thus also relates to the use of a fungal strain according to the invention for producing biofuel from cellulosic or lignocellulosic substrates.

According to the invention, the term “biofuel” is understood more specifically to refer to a second-generation biofuel, i.e. a biofuel derived from non-food resources. According to the invention, the term “biofuel” may also be defined as any product derived from biomass transformation and that can be used for energy purposes. First, without wishing to place any limitations, one may cite as examples biogas, products that can be incorporated (possibly after subsequent transformation) into a fuel or that may be a fully-fledged fuel, such as alcohols (ethanol, butanol and/or isopropanol, depending on the type of fermentative organism used), solvents (acetone), acids (butyric), lipids and their derivatives (short- or long-chain fatty acids, fatty acid esters), as well as hydrogen. Preferably, the biofuel according to the invention is an alcohol, for example ethanol, butanol and/or isopropanol. More preferentially, the biofuel according to the invention is ethanol. In another embodiment, the biofuel is biogas. In another embodiment, the product is a molecule of interest to the chemical industry, for example another alcohol such as 1,2-propane diol, 1,3-propane diol, 1,4-butane diol, 2,3-butane diol, organic acids such as acetic acid, propionic acid, acrylic acid, butyric acid, succinic acid, malic acid, fumaric acid, citric acid, itaconic acid, or hydroxy acids such as glycolic acid, hydroxypropionic acid, or lactic acid.

In a preferred aspect, the method of producing a biofuel from cellulosic or lignocellulosic substrates according to the invention comprises:

i) a step of pretreating a cellulosic or lignocellulosic substrate in order to obtain a pretreated substrate,

ii) a step of using the strain according to the invention to produce cellulolytic enzymes,

iii) a step of enzymatically hydrolyzing the pretreated substrate obtained in step i), in the presence of cellulolytic enzymes obtained in step ii), in order to obtain a hydrolysate.

iv) a step of alcoholic fermentation of the hydrolysate obtained,

v) a separation step, in particular by distillation.

In an even more preferred aspect, the method of producing a biofuel from cellulosic or lignocellulosic substrates according to the invention comprises:

i) a step of pretreating a cellulosic or lignocellulosic substrate in order to obtain a pretreated substrate,

ii) a step of using the strain according to the invention to produce cellulolytic enzymes,

iii) a step of enzymatically hydrolyzing the pretreated substrate obtained in step i), in the presence of cellulolytic enzymes obtained in step ii), in order to obtain a hydrolysate.

iv) a step of alcohol fermentation of the hydrolysate obtained,

v) a separation step, in particular by distillation.

said steps iii) and iv) being carried out simultaneously. This is typically the case in production methods known as “SSF” (Simultaneous Saccharification and Fermentation).

According to one particular embodiment, the step of pretreating a cellulosic or lignocellulosic substrate is a step of suspending said cellulosic or lignocellulosic substrate in aqueous phase.

According to one particular embodiment, the hydrolysate obtained in step iii) is a hydrolysate containing glucose.

According to one particular embodiment, the step of alcoholic fermentation of the hydrolysate obtained is a step of fermenting, in the presence of a fermentative organism, the glucose issued from the hydrolysate so as to produce a fermentation must. A fermentative organism is, for example, a yeast.

According to one particular embodiment, the separation step is a separation of the biofuel and the fermentation must, in particular by distillation.

According to an even more preferred embodiment, the cellulosic or lignocellulosic substrate to be hydrolyzed is suspended in aqueous phase at the quantity of 6 to 40% dry matter, preferably 20 to 30%. The pH is adjusted between 4 and 5.5, preferably between 4.8 and 5.2, and the temperature is adjusted between 40° C. and 60° C., preferably between 45° C. and 50° C. The hydrolysis reaction is initiated by the addition of enzymes acting on the pretreated substrate. The amount of enzymes usually used is from 10 to 30 mg of excreted proteins per gram of pretreated substrate, or less. The reaction generally lasts from 15 to 48 hours. The reaction is monitored by assaying the sugars released, particularly glucose. The sugar solution is separated from the unhydrolyzed solid fraction, essentially composed of lignin, by filtration or centrifugation and is then treated in a fermentation unit.

According to another even more preferred embodiment, when the hydrolysis and fermentation steps are carried out simultaneously, the enzymes and the fermentative organism are added simultaneously and then are incubated at a temperature of between 30° C. and 35° C. to produce a fermentation must. According to this embodiment, the cellulose present in the pretreated substrate is converted into glucose, and at the same time, in the same reactor, the fermentative organism (for example, a yeast) converts the glucose into the final product according to an SSF (Simultaneous Saccharification and Fermentation) process known to the person skilled in the art. Depending on the metabolic and hydrolytic capacities of the fermentative organism, the successful completion of the operation may require the addition of a greater or lesser amount of exogenous cellulolytic mixture.

In a seventh aspect, the invention also relates to the use of a fungal strain according to the invention for the hydrolysis of cellulose or of lignocellulose into glucose.

In an eighth aspect, the invention also relates to the use of a fungal strain according to the invention to improve the properties of a compatible strain, particularly an industrial strain.

In the present description, the definitions and preferences indicated in one aspect apply mutatis mutandis to the other aspects. For example, all definitions and preferences indicated in the first aspect of the invention above also apply to the second, third, fourth, fifth, sixth, seventh, and eighth aspects.

### BRIEF DESCRIPTION OF THE FIGURES

Other features, details and advantages of the invention will appear upon reading the attached Figures.

FIG. 1

FIG. 1 represents the apparent viscosities measured at a shear rate of 5 s⁻¹ for different strains cultivated in shake flasks.

FIG. 2

FIG. 2 represents the apparent viscosities measured at a shear rate of 5 s⁻¹ for different strains cultivated in a bioreactor (RutC30 and TR3126-ΔGEL3)

FIG. 3

FIG. 3 represents the apparent viscosities measured at a shear rate of 5 s⁻¹ for different strains cultivated in a bioreactor (CL847 and CL847-ΔGEL3)

### SEQUENCES OF THE PRESENT INVENTION

TABLE 1

| Name of | |
|---|---|
| the se- | |
| quence | Sequence |
| SEQ ID | gactaagaga gagagagagg gagcattggc ctctatcgtg atgcctcggt gaatagagag agtgagtgtg tgtgtggtat |
| NO: 1 | ttcccactgc tgcgagcttc aattgagagc aatggccatg atattacaca aggttcctac ctaggtaagg taatgcaagg |
| | tactacagcg aggtacagta atactagcac gcaaacaaac atgggccgca acacgagagt cgtaaatccc atcgtctacc |
| | tactgcctac gagtacatgt agcctcgtga ccaactcccc tccccccctt gtcctacgag gctctttgtg cctggacagc |
| | acaggtttcc aaccgtcgca cctcgagcca agaaatccga gccgtttagt agccaattga tgccggatcc tcttcatcct |
| | cgtctggggc actttccacc cacccctgtt tttttttctc ttttcttttt tgcgagctac tgctagtcgc attctggcag |
| | agatggggaa attgaaaggc tagacaggac gatatccttg attttccctg ttcgtatcgt atctggcttg aaaagatggg |
| | ctaacggcaa gggggtctga agcggaacta gtaggacagg gcaggacggg acaggacagg acagggtcaa ggccaaggcc |
| | gaggtcgtgg tgcatgctgg tgtacaagta cgatgccata acaatctcat catcgcgctt ctcttgctgg tggacgggat |
| | ctgcaggttt cctcctcttt tctcttacca ttcttttgac tctctcggtg cctctgcagc cgctctttct tgcattcgcc |
| | ggcttcaatc gcggttgctg gggttattca ctgttggatc caatcactcg ctctttagat aaaacccact ggcttgccag |
| | ctgtcgaggc ccgtccgtcc accgctctcg cttcctctcg tctctctcaa ccctcgctcg ctcgctcgct ctctctctgg |
| | gtcacgttgc gctcactttc ttggttttcc cccctttccg cctctggggg acaatctgtg gccaaacaat tcttgggttg |
| | ggacgagaaa aaaatccgcc ttgtgctcgg ttccctcttt cttttacctt tctctcagtt tcggttttga acccattgac |
| | gagcttctct tcttggttgg tttggatctg ctcgaccaca tcgcgagccc ttcattcatc ccatctttct tttttttttt |
| | ctcttgttcg ccaagagttt tggaacagtg aacagaattc gatttaccgc cgcttccctt tggaaccaac agcttacctg |
| | catttcgact gtgtgacaca cgcaagtgta cctgtgcatt ctgggtaaac gactcatagg agagttgtaa aaaagtttcg |
| | gccggcgtat tgggtgttac ggagcattca ctaggcaacc atgcatcctt actattgtat accatcttag taggaatgat |
| | ttcgaggttt atacctacga tgaatgtgtg tcctgtaggc ttgagagttc aaggaagaaa catgcaatta tctttgcgaa |
| | cccagggctg gtgacggaat tttcatagtc aagctatcag agtaaagaag aggagcatgt caaagtacaa ttagagacaa |
| | atatatagtc gcgtggagcc aagagcggat tcctcagtct cgtaggtctc ttgacgaccg ttgatctgct tgatctcgtc |
| | tcccgaaaat gaaaatagct ctgctaagct attcttctct tcgccggagc ctgaaggcgt tactaggttg cagtcaatgc |
| | attaatgcat tgcagatgag ctgtatctgg aagaggtaaa cccgaaaacg cgttttattc ttgttgacat ggagctatta |
| | aatcactaga aggcactctt tgctgcttgg acaaatgaac gtatcttatc gagatcctga acaccatttg tctcaactcc |
| | ggctagcgaa ttctcgactc attcctttgc cctcggacga gtgctggggc gtcggtttcc |
| | actatcggcg agtacttcta cacagccatc ggtccagacg gccgcgcttc tgcgggcgat ttgtgtacgc ccgacagtcc |
| | cggctccgga tcggacgatt gcgtcgcatc gaccctgcgc ccaagctgca tcatcgaaat tgccgtcaac caagctctga |
| | tagagttggt caagaccaat gcggagcata tacgcccgga gtcgtggcga tcctgcaagc tccggatgcc tccgctcgaa |
| | gtagcgcgtc tgctgctcca tacaagccaa ccacggcctc cagaagaaga tgttggcgac ctcgtattgg gaatccccga |
| | acatcgcctc gctccagtca atgaccgctg ttatgcggcc attgtccgtc aggacattgt tggagccgaa atccgcgtgc |
| | acgaggtgcc ggacttcggg gcagtcctcg gcccaaagca tcagctcatc gagagcctgc gcgacggacg cactgacggt |
| | gtcgtccatc acagtttgcc agtgatacac atggggatca gcaatcgcgc atatgaaatc acgccatgta gtgtattgac |
| | cgattccttg cggtccgaat gggccgaacc cgctcgtctg gctaagatcg gccgcagcga tcgcatccat agcctccgcg |
| | accggttgta gaacagcggg cagttcggtt tcaggcaggt cttgcaacgt gacaccctgt gcacggcggg agatgcaata |
| | ggtcaggctc tcgctaaact ccccaatgtc aagcacttcc ggaatcggga gcgcggccga tgcaaagtgc cgataaacat |
| | aacgatcttt gtagaaacca tcggcgcagc tatttacccg caggacatat ccacgccctc ctacatcgaa gctgaaagca |
| | cgagattctt cgccctccga gagctgcatc aggtcggaga cgctgtcgaa cttttcgatc agaaacttct cgacagacgt |
| | cgcggtgagt tcaggctttt tcatgatggc cctcctaccg gtgatctcag ctgtaggaaa gagaagaagg ttagtagtcg |
| | acatggtggc cctcctatag tgagtcgtat tatactatgc cgatatacta tgccgatgat taattgtcaa cactaggcgc |
| | cggtcacaac tagtagatat cacttacgtg ttgagaggcg gcatgcgata agaggtgtaa ttacctgaga acatcttgtt |
| | gccctgcttt ccgtgcgaaa tactaccggt acttttggga aacaagggaa caggagggcg ctgctgtgcg cggttctgag |
| | tgttcaggat tgaagctgaa gaaggtgctg aggaagcgta gaactgttgc ggacgcgagt tctgagaaga gctgtaccga |
| | ttggtgaaag ccgaagaagt gagttggtgc cctgttgcct ggataatgtt tgcaactcgc tggttctgca gagacggaga |
| | caaatgctgg ctacgatgtt gctgattcag gttgatacct cggtcgagac actgttttgg tttgataggg tggatttggt |
| | tgcagagaag agaaaggaag gtcaaagagg gaaaactggg cggagggaag gattttgtat caggcagcaa actgccactg |
| | cagtggccct ggcagtgccg ggcgaggcac ccacgcacgg ccgcgcaacc ggttggtcct tgcccaccac gaaacccttc |
| | tgaaaggtca gatggaagtg tgcgacagtg cgcgtcccca agccaatgca ggcgccatgc actccccacc cgcaagattc |
| | actgtgcgtt cttattggtt gccgcaaggc cagccaaagg gggaagtatg agtcacagca ccgatacaag aaaattgcag |
| | aactaacata tggatgcgcg cgctattctg tagagctctg ggcaaagcac caatcctgcg ggtcggtaca cacactagca |
| | ctgccattct ggccgtcaag accgaaactg tcagcgtggc caccgagacc ggtaccagca cggcggagag caccgcagac |
| | agcacctctg gcgacagcac gggtgctgcc acgacttcta ccccgaaatc atcatcttcc accacctcta gcgccgaaag |
| | cacggagacg tccaacggca gccccttgga caagcgcgtc actgcgtttg gtgccggcct ggttggcgtc gttttgggtg |
| | ctgcggtctt gttgtaagat ggattcaagt tggttcgttg gatttgtatc gtcaaatcag tatcagatac ccatctactt |
| | ctcgccatgc ttttctgctc tttgtttctt ttttttgatg ttaccaaggg gcttgagctt gttggcctaa attcccccct |
| | aatgctcttt gtatgatggc tgaacgtttg gagaggggat ggttttgttc tatgacattt gccgcgtaac gaggcatgac |
| | tttattatat taatgcatga ggatatgttg tacatgaggt gtgaagagcc cttgcttatt tatggtgtgg tgtggtctga |
| | cgccgaccaa ccctgtcatc gtgttcttgt tattatgcca cgcgctatcc tagctgaaat acgggaaccg ataattgccc |
| | tcgcgttgtg aaccctcgcg ttgtgaaccc ttgcgttgtg aaccctcgaa aaccgtgttt cccccttttc atatatgctt |
| | tttcatcttc tttctatcca atatctgcgg ttttggtttc atctagtgga actgtaatcg taggtgagag tagtgttttt |
| | aggccagtaa tggttatagt tgtgattgtt tctgaagtat tctgattcag cctggtggca ctgatattcg tcgtcgacaa |
| | aggtatctcg gtcgacaaag gtatgctggt cgaggcggtt gtatcgtcgt cgtcagagga ggaagaggac gagtaggtcc |
| | tgacggccga gttgccgacg aagacgaggc tgacggtgaa gatggcgagg acggaggcac agaggaggcc catggccagg |
| | tagcggatgc gcgggtcgta gcggtggggg gaagagcccc agggggtgcg ggtgtgggtt tggcattgga ggttgtcgat |
| | catttatacg gttttttttc tcttgttttg attttgtagg ttgtttgagc ttgattgatg ttgctgaagt atgtggttga |
| | gaatt |
| SEQ ID | ATGCGTTGGTCTTCGGTTGCCGTTGCGCTGGCGAGCGCCAAATCCTTTGCCGTGGCGCTG |
| NO: 2 | GATCCCGTCTCCGTCGTGGGAAACAAGTTCTTCAACAAGGACGGCTCGCAGTTCTTTATCA |
| | AGGGCATTGCGTACCAGCTTGTTCCGCAGGACCCCCTCGTGGACACTGACCAGTGCAAAC |
| | GCGATGCTAAGCTCATGGCCGAGCTCGGAACAAACACCATCCGCGTCTATCACGTCGACC |
| | CGGACGCCGACCATGACGGCTGCATGAGTGCGTTTGACGACGCCGGCATCTACGTCCTTG |
| | CTGATCTGGATACCTTTGATACCTACATTATTCCCCAAAATAACTACTGGAATAAGACAAAGT |
| | TCGACAGGTATGCCGAGGTCCTGGACACGTTCCAAAAGTACGACAACCTCCTGGGCGTCT |
| | TCGTCGGAAACGAGAACATCGCTACTAAAGATGACTCGCCCACGGCGCCCTACCTCAAGG |
| | CTGCTGCCCGCGACATGAAGGCGTACCGCGACGCCCAGGGCTACCGCGAGATCCCCGTC |
| | GGCTACTCGGCCGCCGATATCTTGCAGCTTCGCCCCATGCTCCAGGACTACCTGACCTGC |
| | GGCGGCAACTCGTCCGAGACAGTCGACTTCTTCGCCCTCAACTCGTACTCGTGGTGCGAC |
| | CCCAGCACGTACAAGGAGTCCACCTACGACCAGCTTGAGGCCTACGCCAAGAAATTCCCC |
| | GTGCCCATCTTCCTCTCCGAGACAGGCTGCATCGTTCCCGGCCCTCGCCAGTTCGACGAC |
| | CAGGACGCCATCTTTGGCCCTGAGATGGTCAATGACTGGAGCGGCGCCATCATCTACGAG |
| | TGGATTCAGGAGGAAAACGGCTACGGAATTATAACATATGCCCCAGCCGGCCAAGCTGCT |
| | GGACCCAACGTCGAGGGTGGCTTCCTTCGCAAGGGCACTCCCACGCCAAAGCTGCCCGA |
| | CTTCACCGCGCTCAAGTCCAAGTGGGCGACCAACACCCCTACCGGCGTCAGCCGAGACGA |
| | CTACGACGCAAAGGACGTGTCGACCCGTGCGTGTCCTTCGTCCACTGCCGGCGGCTGGTG |
| | GCAGGTGGATGGCGATGCCAAATTGCCCACCCTGGGCCAG |
| SEQ ID | MRWSSVAVALASAKSFAVALDPVSVVGNKFFNKDGSQFFIKGIAYQLVPQDPLVDTDQCKRDA |
| NO: 3 | KLMAELGTNTIRVYHVDPDADHDGCMSAFDDAGIYVLADLDTFDTYIIPQNNYWNKTKFDRYAE |
| | VLDTFQKYDNLLGVFVGNENIATKDDSPTAPYLKAAARDMKAYRDAQGYREIPVGYSAADILQL |
| | RPMLQDYLTCGGNSSETVDFFALNSYSWCDPSTYKESTYDQLEAYAKKFPVPIFLSETGCIVPG |
| | PRQFDDQDAIFGPEMVNDWSGAIIYEWIQEENGYGIITYAPAGQAAGPNVEGGFLRKGTPTPKL |
| | PDFTALKSKWATNTPTGVSRDDYDAKDVSTRACPSSTAGGWWQVDGDAKLPTLGQ |
| SEQ ID | GACTAAGAGAGAGAGAGAGGGAGC |
| NO: 4 | |
| SEQ ID | AATTCTCAACCACATACTTCAGCA |
| NO: 5 | |
| SEQ ID | AACACCCAATACGCCGGC |
| NO: 6 | |
| SEQ ID | GCTCTGGGCAAAGCACCAA |
| NO: 7 | |
| SEQ ID | GTGCGGGATGCATGAAGACG |
| NO: 8 | |
| SEQ ID | CCACCCTACGATTCTGCAACC |
| NO: 9 | |
| SEQ ID | gactaagaga gagagagagg gagcattggc ctctatcgtg atgcctcggt gaatagagag agtgagtgtg |
| NO: 10 | tgtgtggtat ttcccactgc tgcgagcttc aattgagagc aatggccatg atattacaca aggttcctac |
| | ctaggtaagg taatgcaagg tactacagcg aggtacagta atactagcac gcaaacaaac atgggccgca |
| | acacgagagt cgtaaatccc atcgtctacc tactgcctac gagtacatgt agcctcgtga ccaactcccc |
| | tccccccctt gtcctacgag gctctttgtg cctggacagc acaggtttcc aaccgtcgca cctcgagcca |
| | agaaatccga gccgtttagt agccaattga tgccggatcc tcttcatcct cgtctggggc actttccacc cacccctgtt |
| | tttttttctc ttttcttttt tgcgagctac tgctagtcgc attctggcag ctaacggcaa agatggggaa attgaaaggc |
| | tagacaggac gatatccttg attttccctg ttcgtatcgt atctggcttg aaaagatggg gggggtctga |
| | agcggaacta gtaggacagg gcaggacggg acaggacagg acagggtcaa ggccaaggcc gaggtcgtgg |
| | tgcatgctgg tgtacaagta cgatgccata acaatctcat catcgcgctt ctcttgctgg tggacgggat ctgcaggttt |
| | cctcctcttt tctcttacca ttcttttgac tctctcggtg cctctgcagc cgctctttct tgcattcgcc ggcttcaatc |
| | gcggttgctg gggttattca ctgttggatc caatcactcg ctctttagat aaaacccact ggcttgccag ctgtcgaggc |
| | ccgtccgtcc accgctctcg cttcctctcg tctctctcaa ccctcgctcg ctcgctcgct ctctctctgg gtcacgttgc |
| | gctcactttc ttggttttcc cccctttccg cctctggggg acaatctgtg gccaaacaat tcttgggttg ggacgagaaa |
| | aaaatccgcc ttgtgctcgg ttccctcttt cttttacctt tctctcagtt tcggttttga acccattgac gagcttctct |
| | tcttggttgg tttggatctg ctcgaccaca tcgcgagccc ttcattcatc ccatctttct tttttttttt ctcttgttcg |
| | ccaagagttt tggaacagtg aacagaattc gatttaccgc cgcttccctt tggaaccaac agcttacctg |
| | catttcgact gtgtgacaca cg |
| SEQ ID | caagtgta cctgtgcatt ctgggtaaac gactcatagg agagttgtaa aaaagtttcg geeggegtat tgggtgttac |
| NO: 11 | ggagcattca ctaggcaacc atgcatcctt actattgtat accatcttag taggaatgat ttcgaggttt atacctacga |
| | tgaatgtgtg teetgtagge ttgagagttc aaggaagaaa catgcaatta tctttgcgaa cccagggctg |
| | gtgacggaat tttcatagtc aagctatcag agtaaagaag aggagcatgt caaagtacaa ttagagacaa |
| | atatatagtc gcgtggagcc aagagcggat tcctcagtct cgtaggtctc ttgacgaccg ttgatctgct tgatctcgtc |
| | tcccgaaaat gaaaatagct ctgctaagct attcttctct tcgccggagc ctgaaggcgt tactaggttg |
| | cagtcaatgc attaatgcat tgcagatgag ctgtatctgg aagaggtaaa cccgaaaacg cgttttattc |
| | ttgttgacat ggagctatta aatcactaga aggcactctt tgctgcttgg acaaatgaac gtatcttatc gagatcctga |
| | acaccatttg tctcaactcc ggctagcgaa ttctcgactc attcctttgc cctcggacga gtgctggggc gtcggtttcc |
| | actatcggcg agtacttcta cacagccatc ggtccagacg gccgcgcttc tgcgggcgat ttgtgtacgc |
| | ccgacagtcc cggctccgga tcggacgatt gcgtcgcatc gaccctgcgc ccaagctgca tcatcgaaat |
| | tgccgtcaac caagctctga tagagttggt caagaccaat gcggagcata tacgcccgga gtcgtggcga |
| | tcctgcaagc tccggatgcc tccgctcgaa gtagcgcgtc tgctgctcca tacaagccaa ccacggcctc |
| | cagaagaaga tgttggcgac ctcgtattgg gaatccccga acatcgcctc gctccagtca atgaccgctg |
| | ttatgcggcc attgtccgtc aggacattgt tggagccgaa atccgcgtgc acgaggtgcc ggacttcggg |
| | gcagtcctcg gcccaaagca tcagctcatc gagagcctgc gcgacggacg cactgacggt gtcgtccatc |
| | acagtttgcc agtgatacac atggggatca gcaatcgcgc atatgaaatc acgccatgta gtgtattgac |
| | cgattccttg cggtccgaat gggccgaacc cgctcgtctg gctaagatcg gccgcagcga tcgcatccat |
| | agcctccgcg accggttgta gaacagcggg cagttcggtt tcaggcaggt cttgcaacgt gacaccctgt |
| | gcacggcggg agatgcaata ggtcaggctc tcgctaaact ccccaatgtc aagcacttcc ggaatcggga |
| | gcgcggccga tgcaaagtgc cgataaacat aacgatcttt gtagaaacca tcggcgcagc tatttacccg |
| | caggacatat ccacgccctc ctacatcgaa gctgaaagca cgagattctt cgccctccga gagctgcatc |
| | aggtcggaga cgctgtcgaa cttttcgatc agaaacttct cgacagacgt cgcggtgagt tcaggctttt |
| | tcatgatggc cctcctaccg gtgatctcag ctgtaggaaa gagaagaagg ttagtagtcg acatggtggc |
| | cctcctatag tgagtcgtat tatactatgc cgatatacta tgccgatgat taattgtcaa cactaggcgc cggtcacaac |
| | tagtagatat cacttacgtg ttgagaggcg gcatgcgata agaggtgtaa ttacctgaga acatcttgtt gccctgcttt |
| | ccgtgcgaaa tactaccggt acttttggga aacaagggaa caggagggcg ctgctgtgcg cggttctgag |
| | tgttcaggat tgaagctgaa gaaggtgctg aggaagcgta gaactgttgc ggacgcgagt tetgagaaga |
| | gctgtaccga ttggtgaaag ccgaagaagt gagttggtgc cctgttgcct ggataatgtt tgcaactcgc |
| | tggttctgca gagacggaga caaatgctgg ctacgatgtt gctgattcag gttgatacct cggtcgagac |
| | actgttttgg tttgataggg tggatttggt tgcagagaag agaaaggaag gtcaaagagg gaaaactggg |
| | cggagggaag gattttgtat caggcagcaa actgccactg cagtggccct ggcagtgccg ggcgaggcac |
| | ccacgcacgg ccgcgcaacc ggttggtcct tgcccaccac gaaacccttc tgaaaggtca gatggaagtg |
| | tgcgacagtg cgcgtcccca agccaatgca ggcgccatgc actccccacc cgcaagattc actgtgcgtt |
| | cttattggtt gccgcaaggc cagccaaagg gggaagtatg agtcacagca ccgatacaag aaaattgcag |
| | aactaacata tggatgcgcg cgctattctg tagagctctg ggcaaagcac caatcctgcg ggtcggtaca |
| | cacactagca ctgcc |
| SEQ ID | attct ggccgtcaag accgaaactg tcagcgtggc caccgagacc ggtaccagca cggcggagag |
| NO: 12 | caccgcagac agcacctctg gcgacagcac gggtgctgcc acgacttcta ccccgaaatc atcatcttcc |
| | accacctcta gcgccgaaag cacggagacg tccaacggca gccccttgga caagcgcgtc actgcgtttg |
| | gtgccggcct ggttggcgtc gttttgggtg ctgcggtctt gttgtaagat ggattcaagt tggttcgttg gatttgtatc |
| | gtcaaatcag tatcagatac ccatctactt ctcgccatgc ttttctgctc tttgtttctt ttttttgatg ttaccaaggg |
| | gcttgagctt gttggcctaa attcccccct aatgctcttt gtatgatggc tgaacgtttg gagaggggat ggttttgttc |
| | tatgacattt gccgcgtaac gaggcatgac tttattatat taatgcatga ggatatgttg tacatgaggt gtgaagagcc |
| | cttgcttatt tatggtgtgg tgtggtctga cgccgaccaa ccctgtcatc gtgttcttgt tattatgcca |
| | cgcgctatcc tagctgaaat acgggaaccg ataattgccc tcgcgttgtg aaccctcgcg ttgtgaaccc |
| | ttgcgttgtg aaccctcgaa aaccgtgttt cccccttttc atatatgctt tttcatcttc tttctatcca atatctgcgg |
| | ttttggtttc atctagtgga actgtaatcg taggtgagag tagtgttttt aggccagtaa tggttatagt tgtgattgtt |
| | tctgaagtat tctgattcag cctggtggca ctgatattcg tcgtcgacaa aggtatctcg gtcgacaaag gtatgctggt |
| | cgaggcggtt gtatcgtcgt cgtcagagga ggaagaggac gagtaggtcc tgacggccga gttgccgacg |
| | aagacgaggc tgacggtgaa gatggcgagg acggaggcac agaggaggcc catggccagg tagcggatgc |
| | gcgggtcgta gcggtggggg gaagagcccc agggggtgcg ggtgtgggtt tggcattgga ggttgtcgat |
| | catttatacg gttttttttc tcttgttttg attttgtagg ttgtttgagc ttgattgatg ttgctgaagt atgtggttga |
| | gaatt |

### LIST OF REFERENCES CITED IN THE PRESENT PATENT APPLICATION

Durand H, Clanet M, Tiraby G. Genetic improvement of Trichoderma reesei for large scale cellulase production. Enzyme Microb Technol 1988, 10:341-346.
Guangtao Z, Hartl L, Schuster A, Polak S, Schmoll M, Wang T, Seidl V, Seiboth B., (2009). Gene targeting in a nonhomologous end joining deficient Hypocrea jecorina. J Biotechnol. 139(2):146-51.
Montenecourt, B. S.; Eveleigh, D. E. (1977) Semiquantitative Plate Assay for Determination of Cellulase Production by Trichoderma viride. In: Applied and environmental microbiology, vol. 33, No. 1, p. 178-183
Penttila M, Nevalainen H, Rättö M, Salminen E, Knowles J., (1987). A versatile transformation system for the cellulolytic filamentous fungus Trichoderma reesei. Gene. 61(2):155-64.
Punt, P. J.; van den Hondel, C. A., (1992) Transformation of filamentous fungi based on hygromycin B and phleomycin resistance markers. In: Methods in enzymology, vol. 216, p. 447-457.
Te'o V S, Bergquist P L, Nevalainen K M., (2002). Biolistic transformation of Trichoderma reesei using the Bio-Rad seven barrels Hepta Adaptor system. J Microbiol Methods. 51(3):393-9.

### EXAMPLES

### Example 1a: Invalidation of the ID78713 (GEL3) Gene in a Hyper-Producing Strain

The invalidation cassette for ID78713 (GEL3) is composed of the hygromycin antibiotic resistance gene hph placed under the control of the GPDa promoter and the TRPc terminator (Punt and van den Hondel, 1992), with 5′ and 3′ flanking regions upstream and downstream from the ID78713 (GEL3) gene. This sequence is represented in SEQ ID NO: 1. The DNA cassette was synthesized and the cassette was inserted into a pEX-A plasmid (available, for example, from Addgene). After amplification and extraction of the plasmid, the invalidation cassette was amplified by PCR (Polymerase Chain Reaction) using primers p61 and p62 (see Table 2 below).

TABLE 2

| Primer name | Sequences corresponding to the primer |
|---|---|
| P61 | SEQ ID NO: 4 |
| P62 | SEQ ID NO: 5 |
| P78 | SEQ ID NO: 6 |
| P79 | SEQ ID NO: 7 |
| P91 | SEQ ID NO: 8 |
| P92 | SEQ ID NO: 9 |

Primer Sequences of the Present Invention

The strain used for the transformation is the hyper-producing strain RutC30 (Montenecourt and Eveleigh, 1977) in which the gene KU70 (ID 63200) was invalidated by replacing the coding sequence by the gene encoding the selection marker AmdS (Pentillä et al., 1987). Invalidation of this gene promotes homologous recombination (Guangtao et al., 2009). This strain is called TR3126.

Transformations of the TR3126 strain with the cassette represented by SEQ ID NO: 1 were carried out by Biolistique (Te'o et al., 2002) by using 5 μg of purified cassette. Integration at the locus of the invalidation cassette was verified by PCR with a primer (p91) upstream from the cassette and a primer (p78) in the hph gene (5′ verification) and a primer (p92) downstream from the cassette and a primer (p79) in the hph gene (3′ verification). The strains invalidated by the ID78713 (GEL3) gene thus obtained are called TR3126-ΔGEL3.

### Example 1b: Invalidation of the ID78713 (GEL3) Gene in a Hyper-Producing Strain

A second strain was tested: the CL847 strain (Durand H et al.).

Transformation of the CL847 strain was carried out by the protoplast method (Penttila M et al., doi: 10.1016/0378-1119(87)90110-7). Similarly to example 1a, integration at the locus of the invalidation cassette was verified. The strains invalidated by the ID78713 (GEL3) gene thus obtained are called CL847-ΔGEL3.

### Example 2: Method of Measuring Viscosity

For the rheological measurement, the shaft (rotor) used is a large impeller of stainless steel with a diameter of 38 mm, a height of 32 mm, a pitch of 29 mm, and a ribbon 8 mm in width. This impeller is used with a cup (stator) with an inner diameter of 45 mm and a vertical space between the rotor and the stator of 500 μm. After calibration, the impeller is similar to a Couette cylinder with a radius of 14 mm.

The cup is filled with 70 mL of fermentation must collected from a reactor (shake flask, for example as indicated in Example 3, or bioreactor). The viscosity measurements are carried out by logarithmic shear rate sweeps of between 4 s⁻¹ and 100 s⁻¹, at a temperature of 27° C. This range corresponds to the average shear rates expected on an industrial scale. The sweeps are two-way sweeps (from 4 s⁻¹ to 100 s⁻¹ and then from 100 s⁻¹ to 4 s⁻¹). The rheological measurements have been carried out in duplicate.

All measurements are carried out on the TA Instruments AR 2000 rheometer.

### Example 3: Culture Protocol in Shake Flasks

Shake flask culturings are carried out in Fernbach flasks with a diameter of 19 cm, containing 400 mL of culture medium, seeded with spores of different strains from cryotubes, and incubated at 150 rpm and 30° C. in an Infors Multitron incubator.

The culture medium has the following final composition:
5.6 g/L of (NH₄)₂SO₄
4.4 g/L of K₂HPO₄
0.3 g/L of MgSO₄, 7H₂O
0.15 g/L of CaCl₂, 2H₂O
1 mL/L of trace element solution (FeSO₄: 5 g/L, MnSO₄: 1.4 g/L, ZnSO₄: 1.4 g/L, CoCl₂: 3.7 g/I)
5.85 g/L of BTCA (butane tetracarboxylic acid)
3.0 g/L of KOH in crystals
1.5 g/L of corn steep liquor (for example Roquette Solulys®)
30 g/L of glucose

The pH of the culture medium is adjusted to 6.0 with 30% sodium hydroxide.

The compounds are sterilized for 20 minutes at 121° C. (the glucose is sterilized separately from the other compounds).

Regular 2 mL samples are taken to monitor the residual glucose. Then when the residual glucose is less than 5 g/L (which corresponds to a fungal concentration on the order of 10 g/L), a 100 mL sample is taken to measure precisely the fungal concentration (by filtration and then drying on 1.2 μm filters) and the must viscosity (according to the method described in example 2).

### Example 4: Comparison of Viscosities in Shake Flask Cultures

Two strains were cultivated in duplicate according to the method described in Example 3, and then the viscosity of the fermentation must was characterized according to the method described in Example 2:

The two strains tested are:
The TR3126-ΔGEL3 strain presenting invalidation of the ID78713 (GEL3) gene;
The parent strain TR3126 used as a high-viscosity control

Fungal concentration measurements show that the concentrations are of the same order in all culturing carried out, around 10 g/L of fungus in suspension (see Table 3 below).

TABLE 3

| | Fungal concentration (g/L) | |
|---|---|---|
| Strains | Replicate 1 | Replicate 2 |
| TR3126 | 9.4 | 8.7 |
| TR3126-ΔGEL3 | 8.6 | 11.6 |

Fungal concentration for the strains tested (experiments carried out in duplicate)

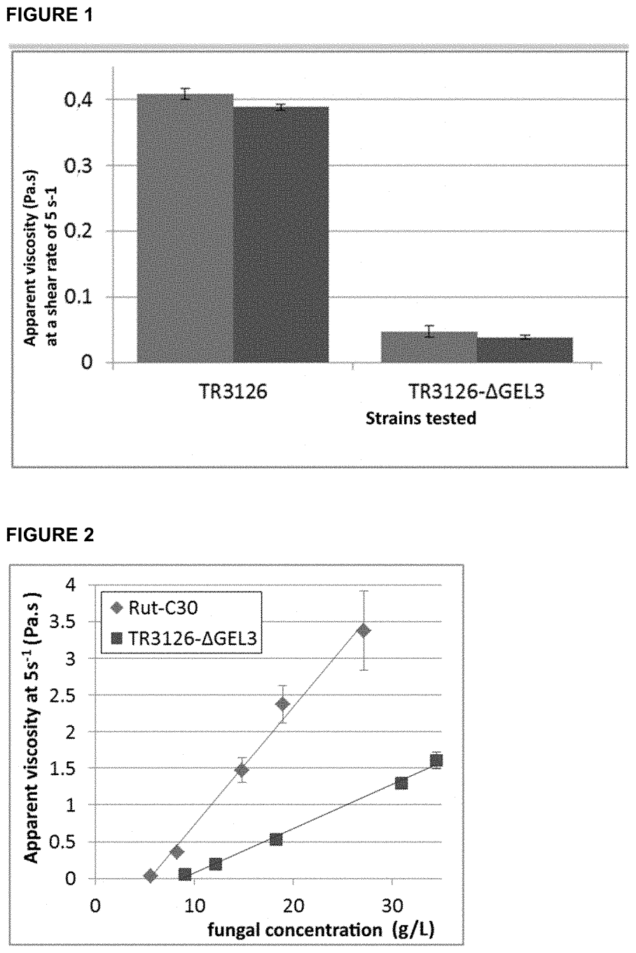

The viscosity measurements (presented in FIG. 1) show that invalidation of the ID78713 (GEL3) gene led to a drastic lowering of viscosity. At a shear rate of 5 s⁻¹, the viscosity obtained for the TR3126-ΔGEL3 strain is approximately 8 to 10 times lower than that of the viscous control (TR3126).

### Example 5: Culture Protocol in Bioreactor

Bioreactor culturings are carried out in fermenters with a diameter of 16 cm, containing 2 L of culture medium, seeded at 10% v/v from a pre-culture done according to the protocol described in Example 3. Agitation is performed by a Rayneri turbine with a diameter of 8 cm at a fixed speed of 1000 rpm. The temperature is controlled at 27° C., and the pH is controlled at 4.8 by the automatic addition of 5N ammonia solution.

The culture medium has the following final composition:
3 mL/L of orthophosphoric acid at 85%
0.25 mL/L of sulfuric acid at 96%
1.66 g/L of potassium hydroxide KOH in crystals
2.8 g/L of (NH₄)₂SO₄
0.6 g/L of MgSO₄, 7H₂O
0.6 g/L of CaCl₂, 2H₂O
0.12 g/L of Na₂HPO₄, 12H₂O
1 mL/L of trace element solution (FeSO₄: 5 g/L, MnSO₄: 1.4 g/L, ZnSO₄: 1.4 g/L, CoCl₂: 3.7 g/I)
1 g/L of corn steep liquor (for example Roquette Solulys®)
80 g/L of glucose

The compounds are sterilized for 20 minutes at 121° C. (the glucose is sterilized separately from the other compounds).

The pH of the culture medium is adjusted and then controlled at 4.8 with the ammonia solution used to check the pH.

Regular samples of approximately 100 mL are taken to: (i) monitor the residual glucose, (ii) precisely measure the fungal concentration (by filtration and then drying on 1.2 μm filters) and (iii) measure the viscosity of the must (according to the method described in Example 2).

### Example 6: Comparison of Viscosities in Bioreactor Cultures

Two strains were cultivated in duplicate according to the method described in Example 5, and the viscosity of the fermentation must was characterized (for different concentrations of fungus in the must) according to the method described in Example 2:
The TR3126-Δ GEL3 strain presenting invalidation of the ID78713 (GEL3) gene
The reference strain Rut-C30 (used as a high-viscosity control)

The characterization of viscosity at different fungal concentrations shows a real advantage conferred by invalidation of the ID78713 (GEL3) gene (see FIG. 2) with:
as in shake flasks, a viscosity approximately 10 times lower when the fungal concentration is on the order of 10 g/L;
a viscosity approximately 3 times lower when the concentration is on the order of 25 g/L;
the viscosity of the strain invalidated for ID78713 (GEL3) (TR3126-ΔGEL3) at 35 g/L of the same order as the viscosity of the wild-type strain (Rut-C30) at 15 g/L.

Thus, a culture of the strain invalidated for ID78713 (GEL3) at 35 g/L does not require more energy for agitation than a culture of the wild-type strain at 15 g/L, which enables the productivity of the culture to be increased at the same energy expenditure.

### Example 7: Comparison of Viscosities of Two Other Strains in Bioreactor Cultures

Two strains were cultivated according to the method described in Example 5, and the viscosity of the fermentation must was characterized (for different concentrations of fungus in the must) according to the method described in Example 2:
The CL847-ΔGEL3 strain presenting invalidation of the ID78713 (GEL3) gene
The parent strain CL847 (used as a control)

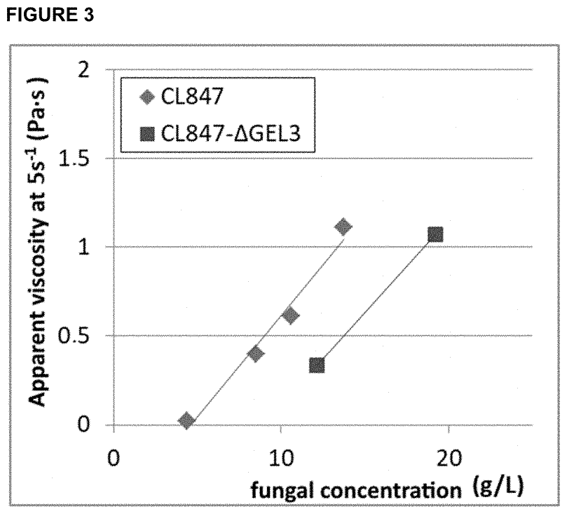

The characterization of the viscosity at different fungal concentrations again shows in the CL847 strain an advantage conferred by invalidation of the ID78713 (GEL3) gene (see FIG. 3) with a viscosity approximately 3 times lower when the fungal concentration is on the order of 12 to 14 g/L.

1-16. (canceled)

17. A strain of fungus wherein the ID78713 gene has been invalidated.

18. The fungal strain according to claim 17, said strain having a reduced viscosity compared to a parent strain wherein the ID78713 (GEL3) gene has not been invalidated.

19. The fungal strain according to claim 17, wherein the ID78713 gene has been invalidated by mutagenesis or by homologous recombination, particularly by using an invalidation cassette represented by SEQ ID NO: 1.

20. The fungal strain according to claim 17, wherein the fungus is a filamentous fungus.

21. The fungal strain according to claim 20, wherein the filamentous fungus belongs to the class of sordariomycetes.

22. The fungal strain according to claim 20, wherein the filamentous fungus belongs to the genus Trichoderma.

23. The fungal strain according to claim 20, wherein the filamentous fungus belongs to the species Trichoderma reesei.

24. The fungal strain according to claim 17, wherein the ID78713 gene corresponds to a gene represented by SEQ ID NO: 2 or a gene having at least 80% identity with the gene of SEQ ID NO: 2, particularly at least 90%, and more specifically at least 95%.

25. A method of genetically modifying a fungal strain according to claim 17, comprising a step of invalidating the ID78713 gene.

26. The method of genetically modifying a fungal strain according to claim 25, wherein the step of invalidating the ID78713 gene is carried out by mutagenesis, by homologous recombination, or more preferentially by using an invalidation cassette represented by SEQ ID NO: 1.

27. A method of producing a fungal biomass, comprising a step of culturing a fungal strain according to claim 17 in a culture medium comprising an appropriate substrate.

28. A method of producing proteins of interest, particularly enzymes, comprising a step of culturing a fungal strain according to claim 17 in a culture medium comprising an appropriate substrate.

29. A method of producing bio-sourced products from cellulosic or lignocellulosic substrates, comprising a step of incubating the fungal strain according to claim 17 under suitable conditions to produce cellulolytic enzymes.

30. A method of producing a biofuel from cellulosic or lignocellulosic substrates, comprising a step of using the fungal strain according to claim 17 to produce cellulolytic enzymes.

31. The method of producing a biofuel from cellulosic or lignocellulosic substrates comprising:
(i) a step of pretreating a cellulosic or lignocellulosic substrate in order to obtain a pretreated substrate,
(ii) a step of using the strain according to claim 1 to produce cellulolytic enzymes,
(iii) a step of enzymatically hydrolyzing the pretreated substrate, in the presence of the cellulolytic enzymes obtained in step (ii) and an appropriate substrate, in order to obtain a hydrolysate,
(iv) a step of alcoholic fermentation of the hydrolysate obtained, step (iv) being optionally carried out simultaneously with step (iii).
(v) a separation step, optionally by distillation.


SEQUENCE LISTING (12 sequences)

SEQ ID NO: 1 - DNA, 5105 bp, Artificial sequence
  Cassette d′invalidation
gactaagagagagagagagggagcattggcctctatcgtgatgcctcggtgaatagagagagtgagtgtgtgtgtggtatttcccactgctgcgagcttcaattgagagcaatggccatgatattacacaaggttcctacctaggtaaggtaatgcaaggtactacagcgaggtacagtaatactagcacgcaaacaaacatgggccgcaacacgagagtcgtaaatcccatcgtctacctactgcctacgagtacatgtagcctcgtgaccaactcccctcccccccttgtcctacgaggctctttgtgcctggacagcacaggtttccaaccgtcgcacctcgagccaagaaatccgagccgtttagtagccaattgatgccggatcctcttcatcctcgtctggggcactttccacccacccctgtttttttttctcttttcttttttgcgagctactgctagtcgcattctggcagctaacggcaaagatggggaaattgaaaggctagacaggacgatatccttgattttccctgttcgtatcgtatctggcttgaaaagatggggggggtctgaagcggaactagtaggacagggcaggacgggacaggacaggacagggtcaaggccaaggccgaggtcgtggtgcatgctggtgtacaagtacgatgccataacaatctcatcatcgcgcttctcttgctggtggacgggatctgcaggtttcctcctcttttctcttaccattcttttgactctctcggtgcctctgcagccgctctttcttgcattcgccggcttcaatcgcggttgctggggttattcactgttggatccaatcactcgctctttagataaaacccactggcttgccagctgtcgaggcccgtccgtccaccgctctcgcttcctctcgtctctctcaaccctcgctcgctcgctcgctctctctctgggtcacgttgcgctcactttcttggttttcccccctttccgcctctgggggacaatctgtggccaaacaattcttgggttgggacgagaaaaaaatccgccttgtgctcggttccctctttcttttacctttctctcagtttcggttttgaacccattgacgagcttctcttcttggttggtttggatctgctcgaccacatcgcgagcccttcattcatcccatctttctttttttttttctcttgttcgccaagagttttggaacagtgaacagaattcgatttaccgccgcttccctttggaaccaacagcttacctgcatttcgactgtgtgacacacgcaagtgtacctgtgcattctgggtaaacgactcataggagagttgtaaaaaagtttcggccggcgtattgggtgttacggagcattcactaggcaaccatgcatccttactattgtataccatcttagtaggaatgatttcgaggtttatacctacgatgaatgtgtgtcctgtaggcttgagagttcaaggaagaaacatgcaattatctttgcgaacccagggctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagctctgctaagctattcttctcttcgccggagcctgaaggcgttactaggttgcagtcaatgcattaatgcattgcagatgagctgtatctggaagaggtaaacccgaaaacgcgttttattcttgttgacatggagctattaaatcactagaaggcactctttgctgcttggacaaatgaacgtatcttatcgagatcctgaacaccatttgtctcaactccggctagcgaattctcgactcattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatgatggccctcctaccggtgatctcagctgtaggaaagagaagaaggttagtagtcgacatggtggccctcctatagtgagtcgtattatactatgccgatatactatgccgatgattaattgtcaacactaggcgccggtcacaactagtagatatcacttacgtgttgagaggcggcatgcgataagaggtgtaattacctgagaacatcttgttgccctgctttccgtgcgaaatactaccggtacttttgggaaacaagggaacaggagggcgctgctgtgcgcggttctgagtgttcaggattgaagctgaagaaggtgctgaggaagcgtagaactgttgcggacgcgagttctgagaagagctgtaccgattggtgaaagccgaagaagtgagttggtgccctgttgcctggataatgtttgcaactcgctggttctgcagagacggagacaaatgctggctacgatgttgctgattcaggttgatacctcggtcgagacactgttttggtttgatagggtggatttggttgcagagaagagaaaggaaggtcaaagagggaaaactgggcggagggaaggattttgtatcaggcagcaaactgccactgcagtggccctggcagtgccgggcgaggcacccacgcacggccgcgcaaccggttggtccttgcccaccacgaaacccttctgaaaggtcagatggaagtgtgcgacagtgcgcgtccccaagccaatgcaggcgccatgcactccccacccgcaagattcactgtgcgttcttattggttgccgcaaggccagccaaagggggaagtatgagtcacagcaccgatacaagaaaattgcagaactaacatatggatgcgcgcgctattctgtagagctctgggcaaagcaccaatcctgcgggtcggtacacacactagcactgccattctggccgtcaagaccgaaactgtcagcgtggccaccgagaccggtaccagcacggcggagagcaccgcagacagcacctctggcgacagcacgggtgctgccacgacttctaccccgaaatcatcatcttccaccacctctagcgccgaaagcacggagacgtccaacggcagccccttggacaagcgcgtcactgcgtttggtgccggcctggttggcgtcgttttgggtgctgcggtcttgttgtaagatggattcaagttggttcgttggatttgtatcgtcaaatcagtatcagatacccatctacttctcgccatgcttttctgctctttgtttcttttttttgatgttaccaaggggcttgagcttgttggcctaaattcccccctaatgctctttgtatgatggctgaacgtttggagaggggatggttttgttctatgacatttgccgcgtaacgaggcatgactttattatattaatgcatgaggatatgttgtacatgaggtgtgaagagcccttgcttatttatggtgtggtgtggtctgacgccgaccaaccctgtcatcgtgttcttgttattatgccacgcgctatcctagctgaaatacgggaaccgataattgccctcgcgttgtgaaccctcgcgttgtgaacccttgcgttgtgaaccctcgaaaaccgtgtttcccccttttcatatatgctttttcatcttctttctatccaatatctgcggttttggtttcatctagtggaactgtaatcgtaggtgagagtagtgtttttaggccagtaatggttatagttgtgattgtttctgaagtattctgattcagcctggtggcactgatattcgtcgtcgacaaaggtatctcggtcgacaaaggtatgctggtcgaggcggttgtatcgtcgtcgtcagaggaggaagaggacgagtaggtcctgacggccgagttgccgacgaagacgaggctgacggtgaagatggcgaggacggaggcacagaggaggcccatggccaggtagcggatgcgcgggtcgtagcggtggggggaagagccccagggggtgcgggtgtgggtttggcattggaggttgtcgatcatttatacggttttttttctcttgttttgattttgtaggttgtttgagcttgattgatgttgctgaagtatgtggttgagaatt

SEQ ID NO: 2 - DNA, 1122 bp, Trichoderma reesei
atgcgttggtcttcggttgccgttgcgctggcgagcgccaaatcctttgccgtggcgctggatcccgtctccgtcgtgggaaacaagttcttcaacaaggacggctcgcagttctttatcaagggcattgcgtaccagcttgttccgcaggaccccctcgtggacactgaccagtgcaaacgcgatgctaagctcatggccgagctcggaacaaacaccatccgcgtctatcacgtcgacccggacgccgaccatgacggctgcatgagtgcgtttgacgacgccggcatctacgtccttgctgatctggatacctttgatacctacattattccccaaaataactactggaataagacaaagttcgacaggtatgccgaggtcctggacacgttccaaaagtacgacaacctcctgggcgtcttcgtcggaaacgagaacatcgctactaaagatgactcgcccacggcgccctacctcaaggctgctgcccgcgacatgaaggcgtaccgcgacgcccagggctaccgcgagatccccgtcggctactcggccgccgatatcttgcagcttcgccccatgctccaggactacctgacctgcggcggcaactcgtccgagacagtcgacttcttcgccctcaactcgtactcgtggtgcgaccccagcacgtacaaggagtccacctacgaccagcttgaggcctacgccaagaaattccccgtgcccatcttcctctccgagacaggctgcatcgttcccggccctcgccagttcgacgaccaggacgccatctttggccctgagatggtcaatgactggagcggcgccatcatctacgagtggattcaggaggaaaacggctacggaattataacatatgccccagccggccaagctgctggacccaacgtcgagggtggcttccttcgcaagggcactcccacgccaaagctgcccgacttcaccgcgctcaagtccaagtgggcgaccaacacccctaccggcgtcagccgagacgactacgacgcaaaggacgtgtcgacccgtgcgtgtccttcgtccactgccggcggctggtggcaggtggatggcgatgccaaattgcccaccctgggccag

SEQ ID NO: 3 - protein, 374 aa, Trichoderma reesei
MRWSSVAVALASAKSFAVALDPVSVVGNKFFNKDGSQFFIKGIAYQLVPQDPLVDTDQCKRDAKLMAELGTNTIRVYHVDPDADHDGCMSAFDDAGIYVLADLDTFDTYIIPQNNYWNKTKFDRYAEVLDTFQKYDNLLGVFVGNENIATKDDSPTAPYLKAAARDMKAYRDAQGYREIPVGYSAADILQLRPMLQDYLTCGGNSSETVDFFALNSYSWCDPSTYKESTYDQLEAYAKKFPVPIFLSETGCIVPGPRQFDDQDAIFGPEMVNDWSGAIIYEWIQEENGYGIITYAPAGQAAGPNVEGGFLRKGTPTPKLPDFTALKSKWATNTPTGVSRDDYDAKDVSTRACPSSTAGGWWQVDGDAKLPTLGQ

SEQ ID NO: 4 - DNA, 24 bp, Artificial sequence
  Amorce
gactaagagagagagagagggagc

SEQ ID NO: 5 - DNA, 24 bp, Artificial sequence
  Amorce
aattctcaaccacatacttcagca

SEQ ID NO: 6 - DNA, 18 bp, Artificial sequence
  Amorce
aacacccaatacgccggc

SEQ ID NO: 7 - DNA, 19 bp, Artificial sequence
  Amorce
gctctgggcaaagcaccaa

SEQ ID NO: 8 - DNA, 20 bp, Artificial sequence
  Amorce
gtgcgggatgcatgaagacg

SEQ ID NO: 9 - DNA, 21 bp, Artificial sequence
  Amorce
ccaccctacgattctgcaacc

SEQ ID NO: 10 - DNA, 1302 bp, Artificial sequence
  Squence 5′ en amont du gne ID78713 (GEL3)
gactaagagagagagagagggagcattggcctctatcgtgatgcctcggtgaatagagagagtgagtgtgtgtgtggtatttcccactgctgcgagcttcaattgagagcaatggccatgatattacacaaggttcctacctaggtaaggtaatgcaaggtactacagcgaggtacagtaatactagcacgcaaacaaacatgggccgcaacacgagagtcgtaaatcccatcgtctacctactgcctacgagtacatgtagcctcgtgaccaactcccctcccccccttgtcctacgaggctctttgtgcctggacagcacaggtttccaaccgtcgcacctcgagccaagaaatccgagccgtttagtagccaattgatgccggatcctcttcatcctcgtctggggcactttccacccacccctgtttttttttctcttttcttttttgcgagctactgctagtcgcattctggcagctaacggcaaagatggggaaattgaaaggctagacaggacgatatccttgattttccctgttcgtatcgtatctggcttgaaaagatggggggggtctgaagcggaactagtaggacagggcaggacgggacaggacaggacagggtcaaggccaaggccgaggtcgtggtgcatgctggtgtacaagtacgatgccataacaatctcatcatcgcgcttctcttgctggtggacgggatctgcaggtttcctcctcttttctcttaccattcttttgactctctcggtgcctctgcagccgctctttcttgcattcgccggcttcaatcgcggttgctggggttattcactgttggatccaatcactcgctctttagataaaacccactggcttgccagctgtcgaggcccgtccgtccaccgctctcgcttcctctcgtctctctcaaccctcgctcgctcgctcgctctctctctgggtcacgttgcgctcactttcttggttttcccccctttccgcctctgggggacaatctgtggccaaacaattcttgggttgggacgagaaaaaaatccgccttgtgctcggttccctctttcttttacctttctctcagtttcggttttgaacccattgacgagcttctcttcttggttggtttggatctgctcgaccacatcgcgagcccttcattcatcccatctttctttttttttttctcttgttcgccaagagttttggaacagtgaacagaattcgatttaccgccgcttccctttggaaccaacagcttacctgcatttcgactgtgtgacacacg

SEQ ID NO: 11 - DNA, 2603 bp, Escherichia coli
caagtgtacctgtgcattctgggtaaacgactcataggagagttgtaaaaaagtttcggccggcgtattgggtgttacggagcattcactaggcaaccatgcatccttactattgtataccatcttagtaggaatgatttcgaggtttatacctacgatgaatgtgtgtcctgtaggcttgagagttcaaggaagaaacatgcaattatctttgcgaacccagggctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagctctgctaagctattcttctcttcgccggagcctgaaggcgttactaggttgcagtcaatgcattaatgcattgcagatgagctgtatctggaagaggtaaacccgaaaacgcgttttattcttgttgacatggagctattaaatcactagaaggcactctttgctgcttggacaaatgaacgtatcttatcgagatcctgaacaccatttgtctcaactccggctagcgaattctcgactcattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatgatggccctcctaccggtgatctcagctgtaggaaagagaagaaggttagtagtcgacatggtggccctcctatagtgagtcgtattatactatgccgatatactatgccgatgattaattgtcaacactaggcgccggtcacaactagtagatatcacttacgtgttgagaggcggcatgcgataagaggtgtaattacctgagaacatcttgttgccctgctttccgtgcgaaatactaccggtacttttgggaaacaagggaacaggagggcgctgctgtgcgcggttctgagtgttcaggattgaagctgaagaaggtgctgaggaagcgtagaactgttgcggacgcgagttctgagaagagctgtaccgattggtgaaagccgaagaagtgagttggtgccctgttgcctggataatgtttgcaactcgctggttctgcagagacggagacaaatgctggctacgatgttgctgattcaggttgatacctcggtcgagacactgttttggtttgatagggtggatttggttgcagagaagagaaaggaaggtcaaagagggaaaactgggcggagggaaggattttgtatcaggcagcaaactgccactgcagtggccctggcagtgccgggcgaggcacccacgcacggccgcgcaaccggttggtccttgcccaccacgaaacccttctgaaaggtcagatggaagtgtgcgacagtgcgcgtccccaagccaatgcaggcgccatgcactccccacccgcaagattcactgtgcgttcttattggttgccgcaaggccagccaaagggggaagtatgagtcacagcaccgatacaagaaaattgcagaactaacatatggatgcgcgcgctattctgtagagctctgggcaaagcaccaatcctgcgggtcggtacacacactagcactgcc

SEQ ID NO: 12 - DNA, 1200 bp, Artificial sequence
  Squence 3′ en aval du gne ID78713 (GEL3)
attctggccgtcaagaccgaaactgtcagcgtggccaccgagaccggtaccagcacggcggagagcaccgcagacagcacctctggcgacagcacgggtgctgccacgacttctaccccgaaatcatcatcttccaccacctctagcgccgaaagcacggagacgtccaacggcagccccttggacaagcgcgtcactgcgtttggtgccggcctggttggcgtcgttttgggtgctgcggtcttgttgtaagatggattcaagttggttcgttggatttgtatcgtcaaatcagtatcagatacccatctacttctcgccatgcttttctgctctttgtttcttttttttgatgttaccaaggggcttgagcttgttggcctaaattcccccctaatgctctttgtatgatggctgaacgtttggagaggggatggttttgttctatgacatttgccgcgtaacgaggcatgactttattatattaatgcatgaggatatgttgtacatgaggtgtgaagagcccttgcttatttatggtgtggtgtggtctgacgccgaccaaccctgtcatcgtgttcttgttattatgccacgcgctatcctagctgaaatacgggaaccgataattgccctcgcgttgtgaaccctcgcgttgtgaacccttgcgttgtgaaccctcgaaaaccgtgtttcccccttttcatatatgctttttcatcttctttctatccaatatctgcggttttggtttcatctagtggaactgtaatcgtaggtgagagtagtgtttttaggccagtaatggttatagttgtgattgtttctgaagtattctgattcagcctggtggcactgatattcgtcgtcgacaaaggtatctcggtcgacaaaggtatgctggtcgaggcggttgtatcgtcgtcgtcagaggaggaagaggacgagtaggtcctgacggccgagttgccgacgaagacgaggctgacggtgaagatggcgaggacggaggcacagaggaggcccatggccaggtagcggatgcgcgggtcgtagcggtggggggaagagccccagggggtgcgggtgtgggtttggcattggaggttgtcgatcatttatacggttttttttctcttgttttgattttgtaggttgtttgagcttgattgatgttgctgaagtatgtggttgagaatt